(19) United States
(12) Patent Application Publication
(10) Pub. No.: US 2026/0257024 A1
(43) Pub. Date: Sep. 3, 2026

(54) A safety device for a medicament delivery device

(71) Applicant: SHL Medical AG, Zug (CH)

(72) Inventor: Anders Boström, Nacka Strand (SE)

(21) Appl. No.: 18/718,496

(22) PCT Filed: Nov. 29, 2022
(86) PCT No.: PCT/EP2022/083695

§ 371 Date: Jun. 11, 2024

(30) Foreign Application Priority Data
Dec. 13, 2021 (EP) 21214087.5

(51) Int. Cl. A61M 5/32

(52) U.S. Cl. CPC A61M 5/3271; A61M 5/3245; A61M 2005/3247

(57) ABSTRACT

The present disclosure generally relates to medicament delivery devices such as autoinjectors, and particularly concerns a safety device adapted to prevent injuries to a user caused by a medicament delivery member at a proximal end of the medicament delivery device.

### CROSS-REFERENCE TO RELATED APPLICATIONS

The present application is a U.S. National Phase Application pursuant to 35 U.S.C. § 371 of International Application No. PCT/EP2022/083695 filed Nov. 29, 2022, which claims priority to European Patent Application No. 21214087.5 filed Dec. 13, 2021. The entire disclosure contents of these applications are herewith incorporated by reference into the present application.

### TECHNICAL FIELD

The present disclosure generally relates to medicament delivery devices such as autoinjectors, and particularly concerns a safety device adapted to prevent injuries to a user after a medicament delivery operation caused by a medicament delivery member at a proximal end of the medicament delivery device.

### BACKGROUND

A number of medical conditions require injections. These days, a number of different injection devices exist, including various types of pen injectors, autoinjectors and on-body devices. Although many of these devices have enabled major improvements in the management of a number of medical conditions, various limitations do still exist in the current technology. Not least amongst these are the difficulties faced by patients that require frequent injections and by patients that need to inject particularly viscous drugs. In considering these problems, the applicant has appreciated that various developments could be made to help improve the medicament delivery devices on the market today, for example concerning preventing injuries to a user caused by a needle of the medicament delivery devices, which are set out in more detail below.

### SUMMARY

An object of the present disclosure is to provide a safety device for a medicament delivery device which solves, or at least mitigates problems of the prior art.

According to a first aspect of the present disclosure, there is provided a safety device for a medicament delivery device.

The safety device comprising a housing extending in a longitudinal direction from a proximal end to a distal end relative a longitudinal axis;
a cover structure configured to surround a medicament delivery member at the proximal end of the housing, the cover structure is axially movable in relation to the housing along the longitudinal axis between a retracted position in which the medicament delivery member is exposed at the proximal end of the cover structure, and an extended position with the medicament delivery member covered by the cover structure;
a carrier immovably arranged within the housing configured to hold the medicament delivery member, the carrier comprises a stop surface facing the proximal end of the housing; and
a lock member arranged coaxially inside the cover structure within the housing, wherein the lock member comprises a distally facing surface, and an engagement element comprising a proximally facing surface, wherein a motion of the cover structure in a proximal direction towards the extended position after a medicament delivery operation causes a transition from an initial position where the proximally facing surface of the engagement element is spaced apart from a distally facing surface of the cover structure to a locked position where the proximally facing surface of the engagement element abuts the distally facing surface of the cover structure,
wherein, in the locked position, a distal motion of the lock member along the longitudinal axis of the housing is blocked by the stop surface of the carrier abutting the distally facing surface of the lock member.

Embodiments of the present disclosure advantageously provides for a lockout function for the cover structure so that once a user has used the medicament delivery device for a medicament delivery operation, thus having pushed the cover structure to the retracted position, and subsequently released to the extended position, the lock member has reached a locking position where the proximally facing surface of the engagement element abuts the distally facing surface of the cover structure, and a distal motion of the lock member along the longitudinal axis of the housing is blocked by the stop surface of the carrier. In this way, the cover structure is locked from moving distally in relation to the housing after a medicament delivery operation.

In other words, the lock member serves to maintain the cover structure in the extended position once a medicament delivery operation has been finalized such that a user is prevented from sharp injury caused by a medicament delivery member, such as a needle.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

When the wording ‘at the injection site’ or ‘at the dose delivery site’ is used in this application, it generally refers to the point where the medicament delivery device (e.g., a needle) enters the patient, along with the surrounding area, for example the area where a pad is attached.

According to one embodiment, the lock member may comprise a protrusion protruding outwards in a radial direction with respect to the longitudinal axis of the housing to engage with a guide of the cover structure. Engagement with the guide may advantageously provide for causing a subsequent motion of the lock member when the cover structure moves along the longitudinal axis.

According to one embodiment, the protrusion may be configured to move into engagement with the guide when the cover structure moves to the retracted position. The protrusion may firstly be disengaged from a cut out or recess of the cover structure, and once the cover structure moves to the retracted position, the protrusion reaches the guide. This advantageously ensures that the motion of the lock member caused by the protrusion interacting with the guide occurs only once the cover structure has firstly reached the retracted position for a medicament delivery operation.

According to one embodiment, guide may be helical; and wherein the protrusion of the lock member is configured to move into engagement with the helical track of the guide. The helical track is shaped so that when the protrusion travels in the helical track when the cover structure moves in a proximal direction from the retracted position towards the extended position, the lock member rotates with respect to the cover structure about the longitudinal axis of the housing. The lock member may also rotate with respect to the housing about the longitudinal axis.

According to one embodiment, the protrusion may initially be in a recess or cut-out of the cover structure, wherein the protrusion moves from the recess or cut-out to the helical track when the cover structure initially moves in a distal direction from the extended position to the retracted position for a medicament delivery operation.

According to one embodiment, the protrusion may comprise a proximally facing surface that is inclined with respect to the longitudinal axis of the housing.

According to one embodiment, the engagement element may be a stop rib that comprises the proximally facing surface and the distally facing surface, wherein in the locked position, the stop rib is positioned between the stop surface of the carrier and the distally facing surface of the cover structure. The stop rib may rotate with the lock member to move from being away from the position between the stop surface of the carrier and the distally facing surface of the cover structure to being between the stop surface of the carrier and the distally facing surface of the cover structure. The stop rib may be flexible.

According to one embodiment, the guide may be a groove or a ledge.

According to one embodiment, the engagement element may comprise a protrusion, radially extending with respect to the longitudinal axis, wherein in the initial position the protrusion is arranged in a first recess or cut-out of the cover structure, and in the locked position the protrusion is arranged in a second recess or cut-out comprising the distally receiving surface of the cover structure, the second recess or cut-out being distally arranged with respect to the first recess or cut-out. Thus, part of the transition from the initial position to the locked position is that the protrusion switches recess or cut-out where it is arranged, thereby providing for locking the cover structure to the lock member in two different linearly spaced apart positions.

According to one embodiment, in the initial position the protrusion of the engagement element may be engaged in the first recess or cut-out of the cover structure so that when the cover structure initially moves towards the retracted position, the lock member moves linearly in a distal direction along the longitudinal axis of the housing together with the cover structure.

According to one embodiment, when the cover structure reaches the retracted position, a distal end of the lock member may engage with a distal facing surface of the carrier. In this way, the lock member is locked against being moved linearly in the proximal direction.

According to one embodiment, the distal end of the lock member may comprise a proximally facing surface that abuts against the distal facing surface of the carrier, when the cover structure moves in the proximal direction towards the extended position after the medicament delivery operation. This advantageously provides for a strong locked engagement between the lock member and the protrusion of the carrier for preventing the lock member against being moved linearly in the proximal direction.

According to one embodiment, the distal end of the lock member may advantageously define the distally facing surface of the lock member that abuts against the stop surface in the locked position.

According to one embodiment, the protrusion of the engagement element may comprise a distally facing surface that is inclined with respect to the longitudinal axis of the housing. This advantageously provides for the protrusions to more easily disengage i.e., requiring less force, from the first recess or cut-out when the cover structure moves from the extended position to the retracted position.

There is further provided a medicament delivery device comprising the safety device of any of the herein disclosed embodiments.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an autoinjector according to embodiments of the present disclosure;

FIG. 2A illustrates medicament delivery device with the front cap removed according to embodiments of the present disclosure;

FIG. 2B illustrates medicament delivery device with the cover structure in a retracted position according to embodiments of the present disclosure;

FIG. 2C illustrates medicament delivery device with the cover structure in an extended position after a medicament delivery operation according to embodiments of the present disclosure;

FIG. 3 is an exploded view of a safety device according to embodiments of the present disclosure;

FIG. 4 illustrates a carrier according to embodiments of the present disclosure;

FIG. 5A-B are different perspective views of a lock member according to embodiments of the present disclosure;

FIG. 6A-B are different perspective views of a cover structure according to embodiments of the present disclosure;

FIG. 7A-C illustrates illustrate a transition from an initial state to a locked state for the safety device according to embodiments of the present disclosure;

FIG. 8 is a close up view of the engagement element arranged between the stop surface of the carrier and the distally facing surface of the cover structure in the locked position according to embodiments of the present disclosure;

FIG. 9 is an exploded view of a safety device according to embodiments of the present disclosure;

FIG. 10 is a perspective views of a cover structure according to embodiments of the present disclosure;

FIG. 11 illustrates a carrier according to embodiments of the present disclosure;

FIG. 12A-B are different perspective views of a lock member according to embodiments of the present disclosure;

FIG. 13A-C illustrates illustrate a transition from an initial state to a locked state for the safety device according to embodiments of the present disclosure; and

FIG. 14 is a close-up view of an engagement between a distal end of the lock member and a distal facing surface of the carrier according to embodiments of the present disclosure.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

In the Figures, the longitudinal direction is the direction of axis 102, with the corresponding circumferential direction 31 and radial direction 33 relative to the axis 102 also shown.

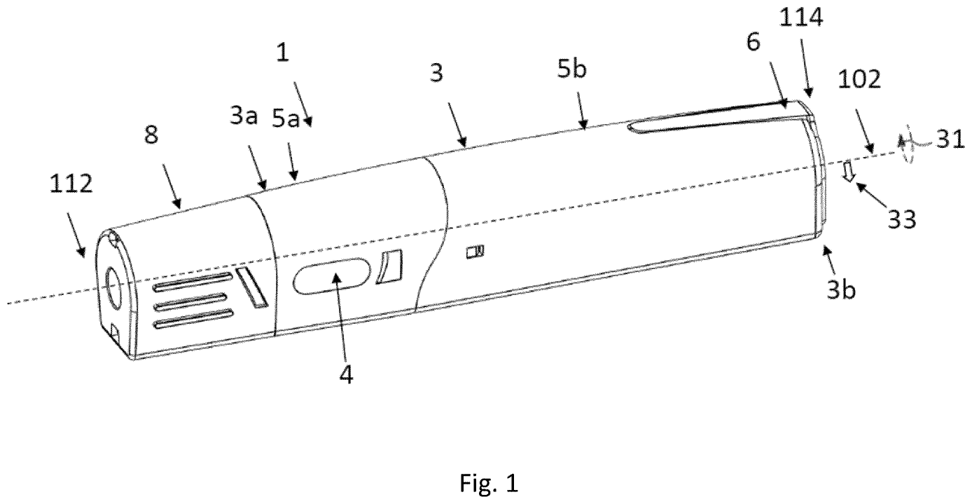

FIG. 1 shows an example of a medicament delivery device 1 such as an autoinjector according to embodiments of the present disclosure. The medicament delivery device 1 is configured to expel medicament from a medicament container via a medicament delivery member, e.g. a needle or a nozzle, to a user at a dose delivery site. The medicament delivery device 1 extends from a proximal end 112 to a distal end 114 relative to the axis 102.

The medicament delivery device 1 comprises a housing 3 with a window 4. The housing 3 has a proximal end 3a and a distal end 3b, and a proximal housing part 5a and a distal housing part 5b. A safety clip 6 is arranged at the distal end 3b of the housing and is removed before use of the medicament delivery device 1. A front cap 8 is arranged at proximal end 3a of the housing 3 to cover a cover structure.

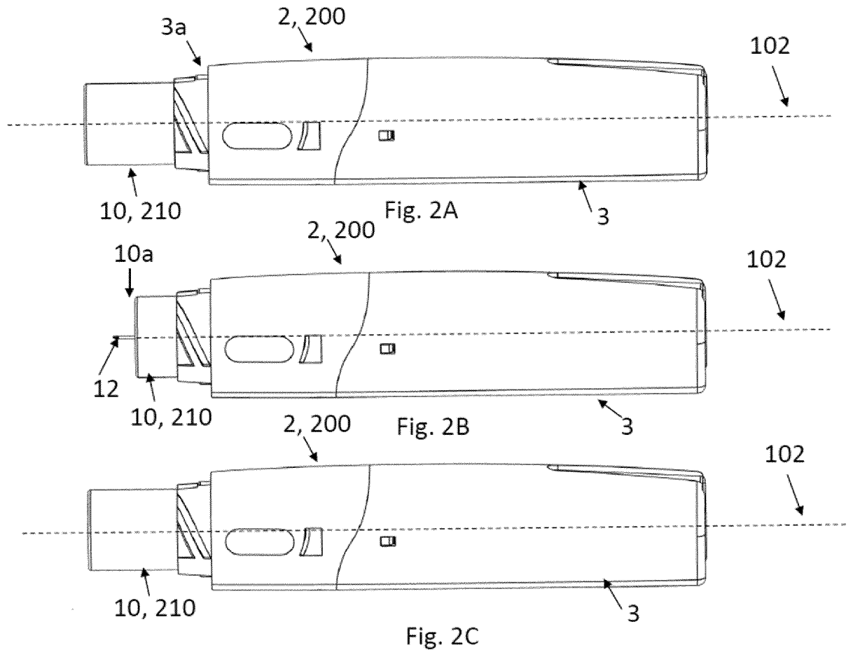

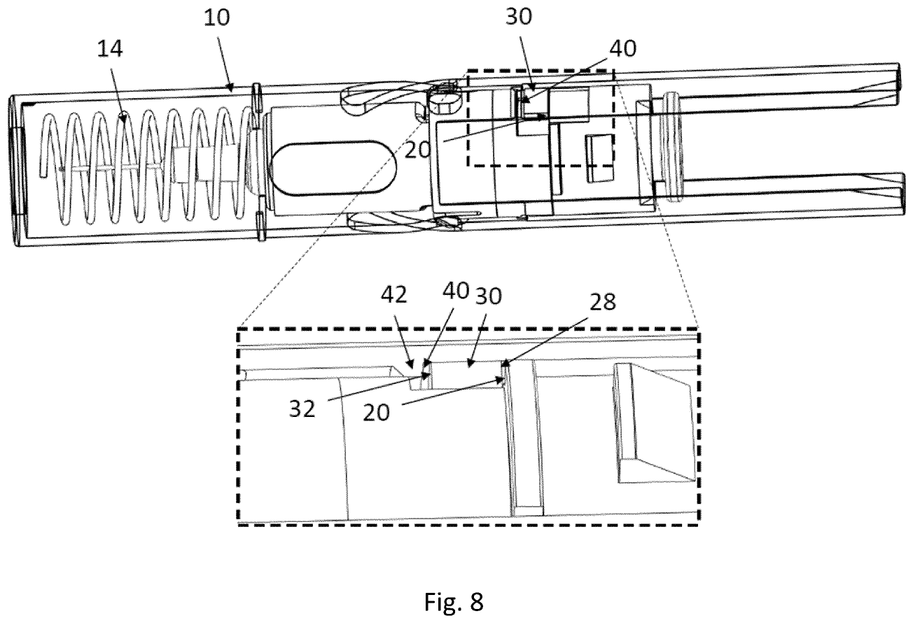

FIG. 2A illustrates the medicament delivery device with the front cap 8 removed so that the cover structure 10 is exposed. The cover structure 10 is configured to surround the medicament delivery member, such as a needle 12 at the proximal end 3a of the housing. The extended position is considered a proximal position of the cover structure 10. The cover structure 10 is biased by e.g., a spring 14 (see FIG. 8) in the proximal direction towards the extended position.

In FIG. 2B, the cover structure 10 has been axially moved in relation to the housing 3 along the longitudinal axis 102 to a retracted position where the cover structure 10 received further in the housing 3 and in which the needle 12 is exposed at the proximal end 10a of the cover structure 10.

After a medicament delivery operation, the cover structure 10 is returned to the extended position, again shown in FIG. 2C, to thereby cover the needle 12 so that a sharp injury can be prevented.

It would be desirable to ensure that the cover structure 10 maintains in the extended position shown in FIG. 2C after a medicament delivery operation to reduce the risk of injury caused by the needle 12. To achieve this, the present disclosure discloses a safety device that will now be described in more detail with reference to subsequent drawings.

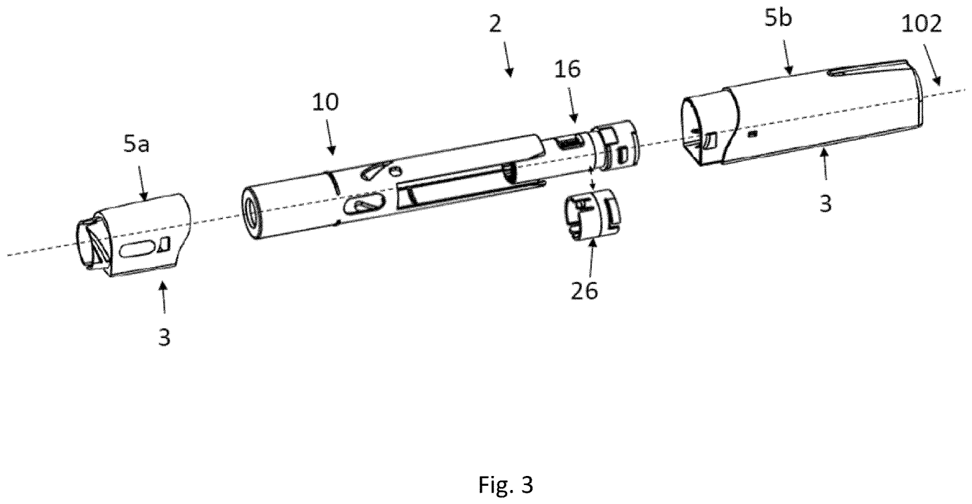

FIG. 3 is an exploded view of the safety device 2 which comprises a housing 3, a cover structure 10, a carrier 16 for holding a medicament delivery member 12, and a lock member 26. The carrier 16, the cover structure 10 and the lock member 26 are arranged in the housing 3 which comprises a proximal housing part 5a and a distal housing part 5b. The carrier is arranged coaxially inside the cover structure and is immovable with respect to the housing 3. The lock member 26 is arranged coaxially to surround the circumference of the carrier. The lock member 26 is rotatable about the axis 102 with respect to the carrier 16 and the cover structure 10. The separate parts of the safety device 2 will now be describe in more detail.

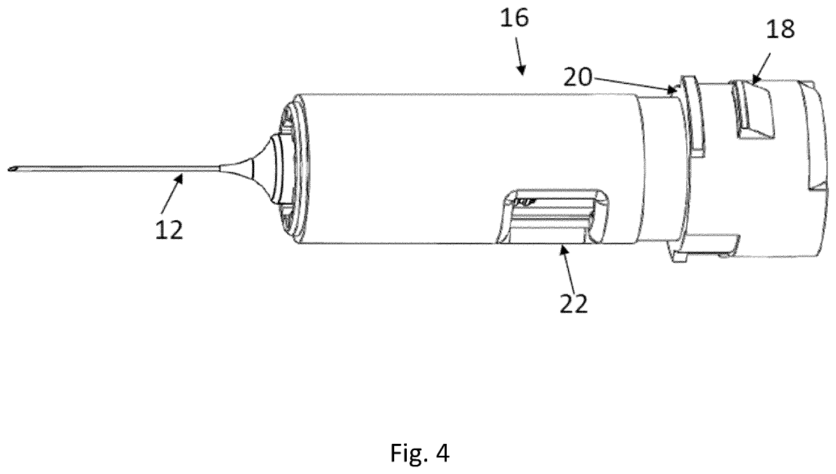

Turning to FIG. 4, illustrating the carrier 16 configured to hold the medicament delivery member 12. The carrier 16 has a generally cylindrical body and is arrange in the housing 3 in such a way that it is immovable in the housing. For example, the protrusion 18 may engage with a receiving feature on either the housing 3 or on another part of the medicament delivery device 1. The carrier 16 comprises a stop surface 20 facing the proximal end 3b of the housing 3. In a preferred example, the cover structure 10 comprises a proximal cover section configured to surround the medicament delivery member and a distal body section. In a preferred example, the distal body section is formed by two arms extending from the proximal cover section towards the distal end of the housing 3. In a preferred example, the stop surface 20 is snugly fit between the two arms of the cover structure 10. In this example, the carrier can be assembled along the two arms of the cover structure.

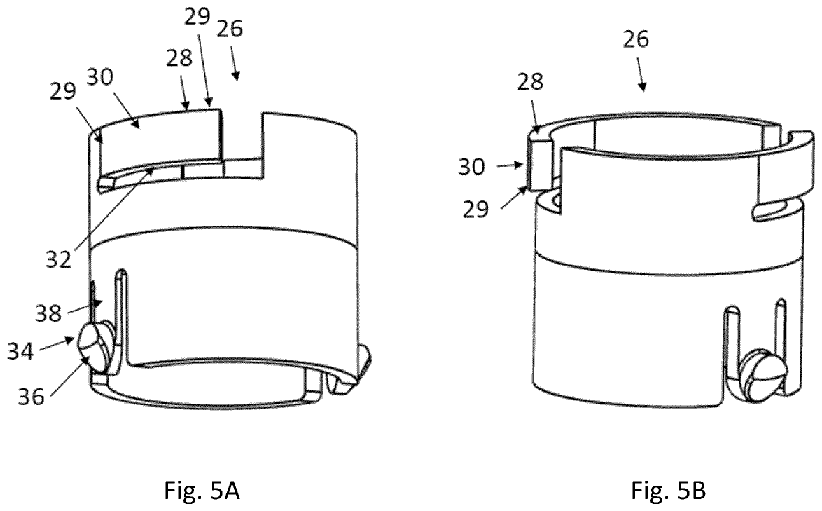

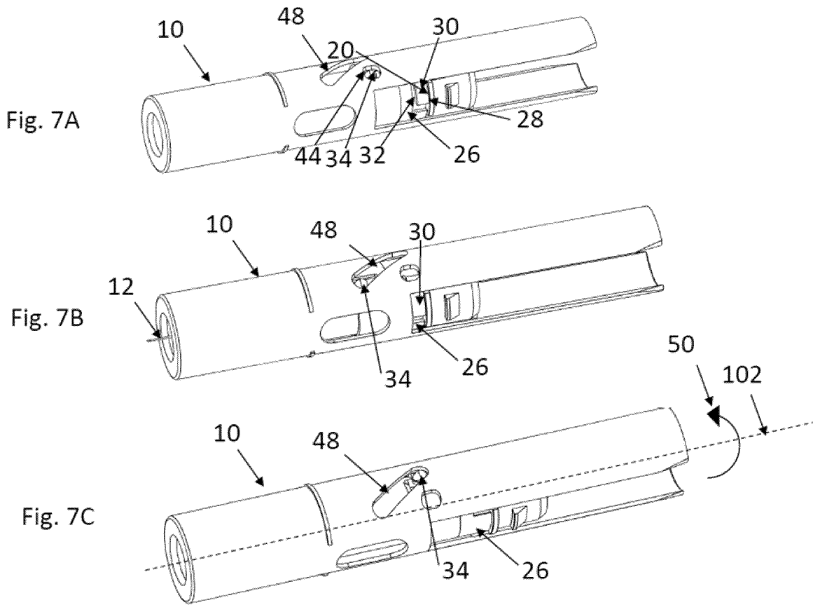

FIG. 5A and FIG. 5B illustrates two different views of a lock member 26. As is better seen in e.g., FIGS. 7A-C, the lock member 26 is arranged coaxially inside the cover structure within the housing 3.

The lock member 26 comprises a distally facing surface 28, and an engagement element 30 comprising a proximally facing surface 32. The lock member 26 further comprises a protrusion 34 protruding outwards in a radial direction with respect to the longitudinal axis of the safety device.

The protrusion 34 comprises a proximally facing surface 36 that is inclined with respect to the longitudinal axis 102 of the housing 3.

The engagement element 30 is shaped as a rib and may be made from a cut-out in the body of the lock member 26. The engagement element 30 thus comprises a free end 27 and a fixed end 29 with respect to the body of the lock member 26. The engagement element 30 extends gradually radially further away from the longitudinal axis 102 from the fixed end 29 to the free end 27. As best seen in FIG. 5B, the free end 29 of engagement element 30 is arranged radially outside the main body of the lock member 26 with respect to the longitudinal axis 102. The engagement element 30 is flexible and can flex towards the longitudinal axis 102, which is useful for the lockout function provided by the herein described safety device and which will be described in more detail with reference to FIGS. 7A-C.

The engagement element 30 in the form of a rib comprises a proximally facing surface 32 and the distally facing surface 28.

The protrusion 34 is arranged at a proximal end of a flexible arm 38. The arm may flex radially with respect to the longitudinal axis 102.

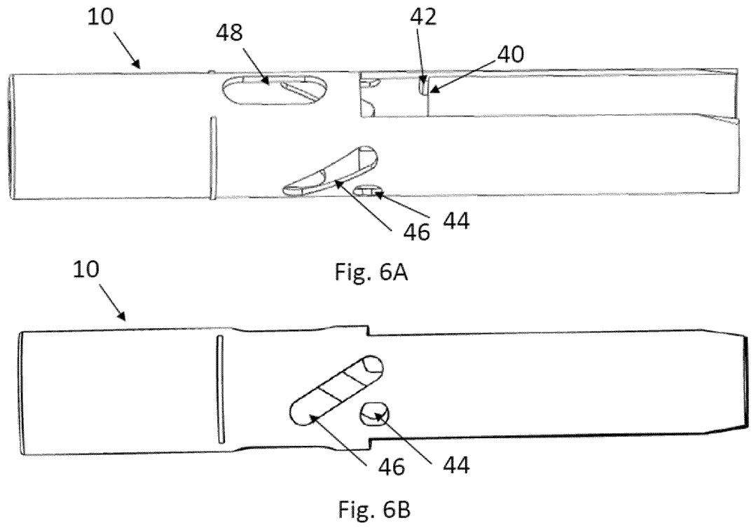

FIG. 6A-B illustrates two different views of the cover structure 10. The cover structure 10 comprises a distally facing surface 40 formed on an inner side facing towards the longitudinal axis 102 of the housing. The distally facing surface 40 is here part of a protrusion 42 but may equally be made as part of other structures forming ledge or similar.

The cover structure 10 comprises a cut out 44 in which the protrusion 34 of the lock member 26 is adapted to be arranged when the cover structure 10 is in the initial extended position before a medicament delivery operation. The cut-out may equally well be a recess.

The cover structure 10 further comprises a guide in the form of a helical track 46. The helical track 46 extends along a curved path circumferentially around of the body of the cylindrically shaped cover structure 10. The cover structure 10 further comprises a window 48.

FIG. 7A-C illustrate a transition from an initial state to a locked state for the safety device 2.

In FIG. 7A, the cover structure 10 is the extended position. In this initial position the proximally facing surface 32 of the engagement element 30 is spaced apart from a distally facing surface 40 of the cover structure 10. The distally facing surface better seen in FIG. 6A.

The protrusion 34 of the lock member 26 is engaged in the cut out 44. When the cover structure is moved distally in relation to the housing, the protrusion 34 moves out from engagement with the cut out 44 and into engagement with the helical track 48 at a location linearly proximal to the location of the cut out 44.

As described with reference to FIGS. 5A-B, the protrusion 34 comprises an inclined proximally facing surface 36. The inclined surface facilitates for forcing the protrusion 34 out of engagement with the cut out 44 by causing the flexible arm 38 to flex towards the longitudinal axis 102 and thereby moving the protrusion towards the longitudinal axis 102 and out from the cut-out 44.

FIG. 7B illustrates the cover structure in the retracted position with the needle 12 exposed. The distal motion of the cover structure has caused the protrusion 34 to move into engagement with the helical guide track 48. In other words, the protrusion 34 is initially in the cut-out 44 of the cover structure 10. The protrusion 34 moves from the cut-out 44 to the helical track 48 when the cover structure 10 initially moves in a distal direction from the extended position to the retracted position for a medicament delivery operation. The medicament delivery operation at an injection site is performed with the cover structure 10 in the retracted position.

FIG. 7C conceptually illustrates the cover structure after it has moved to the extended position after a medicament delivery operation. This motion of the cover structure 10 in the proximal direction towards the extended position after a medicament delivery operation causes a transition from the initial position the proximally facing surface 32 of the engagement element 30 is spaced apart from a distally facing surface 40 of the cover structure 10 to a locked position.

The protrusion 34 travels in the helical track 48 when the cover structure 10 moves in a proximal direction from the retracted position to the extended position. The cover structure 10 moves linearly along the longitudinal axis 102 of the housing whereby the helical track causes the lock member 26 to rotate, as indicated by arrow 50, with respect to the cover structure 10 about the longitudinal axis 102 of the housing. The lock member 26 may also rotate with respect to the housing.

Turning to FIG. 8, in the locked position, the proximally facing surface 32 of the engagement element 30 abuts the distally facing surface 40 of the cover structure 10. Further, a distal motion of the lock member 26 along the longitudinal axis 102 of the housing is blocked by the stop surface 20 of the carrier 16 abutting the distally facing surface 28 of the lock member 26.

The engagement element 30 is a here a flexible stop rib that comprises the proximally facing surface 32 and the distally facing surface 28. In the locked position the stop rib 30 is positioned between the stop surface 20 of the carrier 16 and the distally facing surface 40 of the cover structure 10, to thereby lock the cover structure in the extended position after a medicament delivery operation. As is better seen in FIG. 7B-C, when the lock member rotates about the longitudinal axis due to the interaction between the helical track 48 and the protrusion, the engagement element moves in under the cover structure to a position radially inside the cover structure 10 with respect to the axis 102.

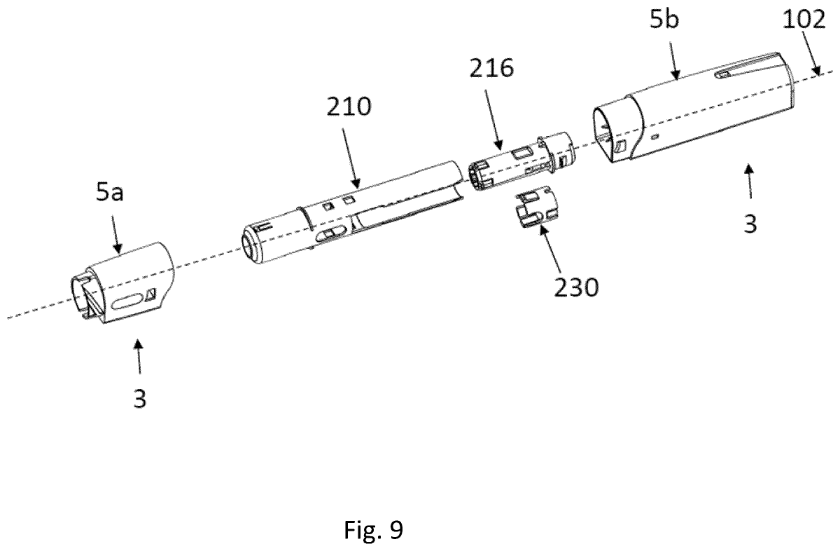

Now turning to another embodiment of the present disclosure. An exploded view of the safety device 200 according to this embodiment is shown in FIG. 9, and comprises a housing 3 as described above, a cover structure 210, a carrier 216, and a lock structure 230, which will now be described in more detail.

FIG. 9 is an exploded view of safety device 200 according to a further embodiment. The safety device 200 comprises a housing 3, a cover structure 210, a carrier 216 for holding a medicament delivery member, and a lock member 230. The carrier 216, the cover structure 210 and the lock member 230 are arranged in the housing 3 which comprises a proximal housing part 5a and a distal housing part 5b. The carrier 216 is arranged coaxially inside the cover structure 210 and is immovable with respect to the housing 3. The lock member 230 is arranged coaxially to surround the circumference of the carrier 216. The lock member is linearly displaceable along the longitudinal axis 102 with respect to the housing 3. The separate parts of the safety device 200 will now be describe in more detail.

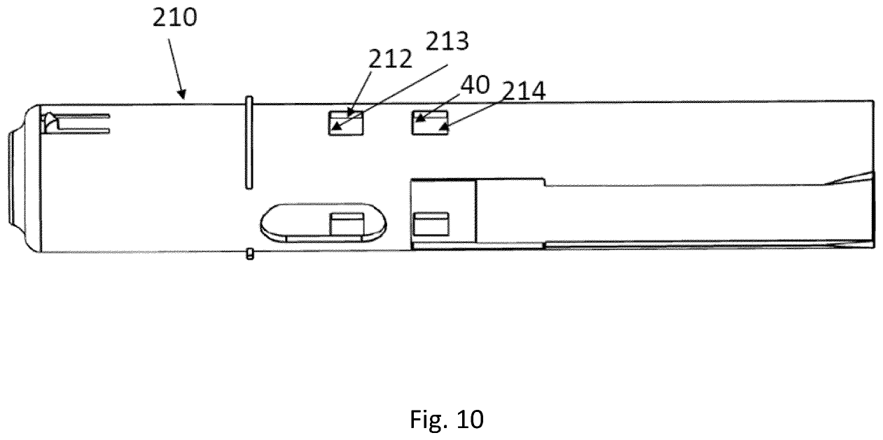

FIG. 10 is a perspective view of the cover structure 210 which comprises a distally facing surface 40. The cover structure 210 comprise a first cut-out 212 and a second cut-out 214. The distally facing surface 40 is part of the perimeter or edges of the second cut-out 214. Further, the first cut-out comprises a proximal surface 213. The first cut-out 212 and the second cut-out 214 may equally well be recesses.

The second cut-out 214 is distally located along the longitudinal axis 102 of the housing with respect to the first cut-out 212.

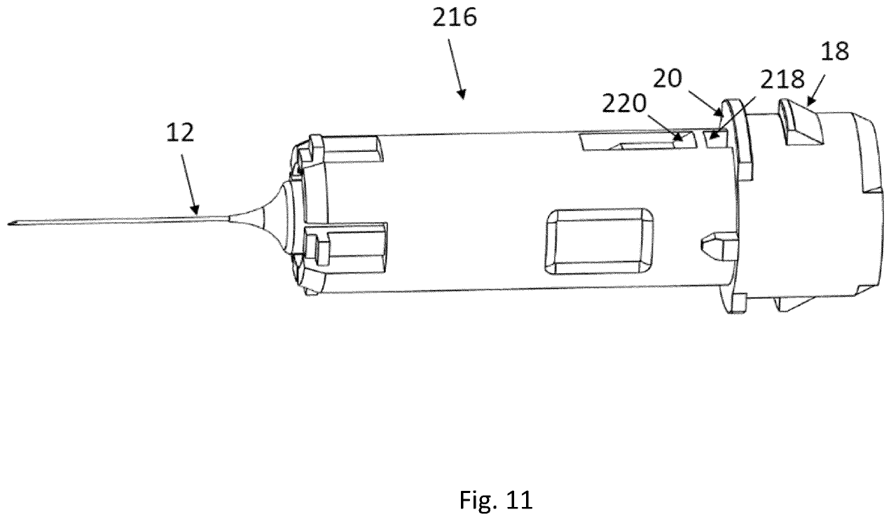

FIG. 11 illustrates a carrier 216 configured to be immovably arranged within the housing 3 and to hold the medicament delivery member 12. The carrier 216 comprises a stop surface 20 facing the proximal end 3a of the housing 3.

The carrier 216 comprises a distal facing surface 218 here formed in a recess of the carrier 216. The distal facing surface 218 may equally well be formed by a protrusion on the carrier 216. Further, proximal to the distal facing surface 218 is an inclined surface 220 defined facing in the proximal direction. The inclination being with respect to the longitudinal axis 102 of the housing 3. As will be described further below, a distal end of the lock member may slide on this inclined surface.

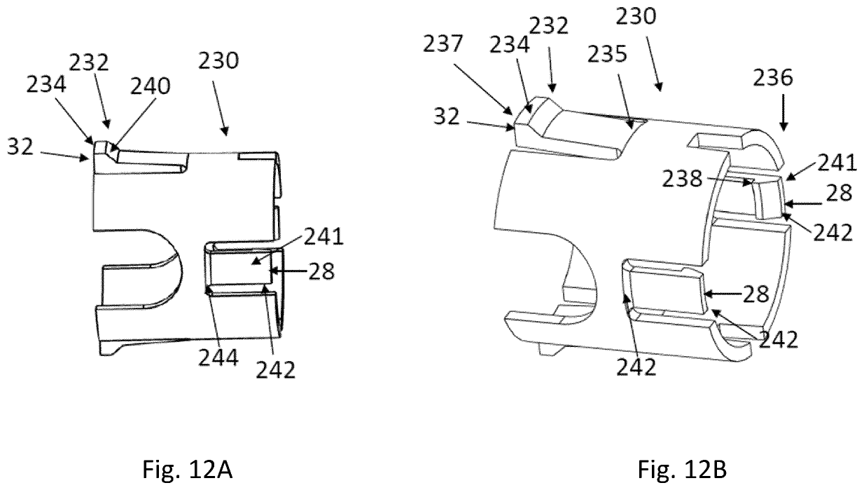

FIGS. 12A-B are perspective view of a lock member 230 according to embodiments of the present disclosure. The lock member 230 is configured to be arranged coaxially inside the cover structure 210 within the housing 3. The lock member 230 comprises a distally facing surface 28, and an engagement element 232 comprising a proximally facing surface 32.

The engagement element 232 comprises a protrusion 234, radially extending with respect to the longitudinal axis 102. The protrusion 234 of the engagement element 232 comprises a distally facing surface 240 that is inclined with respect to the longitudinal axis 102 of the housing.

The engagement element 232 is made from a cut-out having a fixed end 235 and a free end 237 with respect to the body of the lock member 230, where the protrusion 234 is arranged at the free end so that the protrusion can flex in a radial direction with respect to the longitudinal axis 102.

Further, a distal end 236 of the lock member 230 comprises a proximally facing surface 238. The distal end 236 may comprise a cut-through for defining a rib 240 comprising the proximally facing surface 238 and the distally facing surface 28. The rib 240 has a free end 242 and a fixed end 244 with respect to the body of the lock member 230 which allow the rib 240 to flex radially with respect to the longitudinal axis 102.

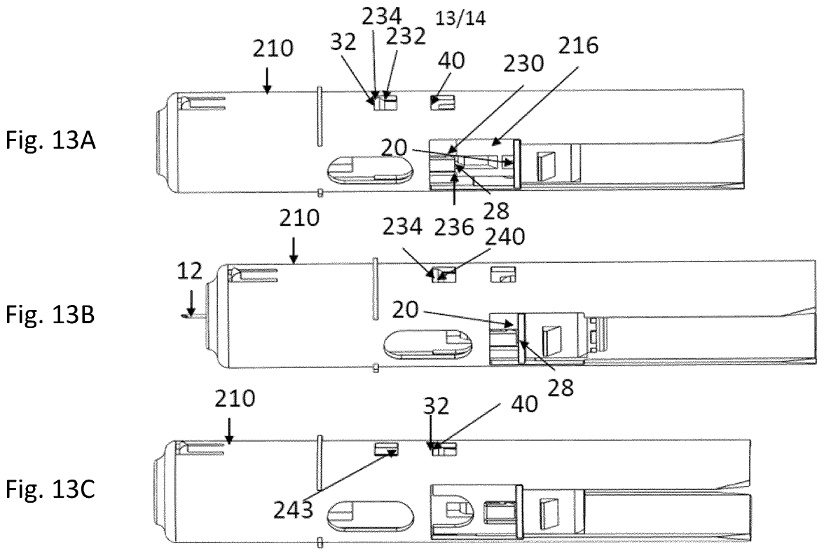

FIG. 13A-C illustrate a transition from an initial state to a locked state for the safety device 200.

In FIG. 13A, the cover structure 210 is the extended position. In this initial position the proximally facing surface 32 of the engagement element 232 is spaced apart from a distally facing surface 40 of the cover structure 210.

In this initial position the protrusion 234 of the engagement element 234 is arranged in the first cut-out 212 of the cover structure 210. The distally facing surface 32 of the engagement element 234 may abut against a proximal surface of the first cut-out 212.

Turning to FIG. 13B, showing the cover structure 210 having moved linearly along the longitudinal axis 102 towards the retracted position, the lock member 230 has also moved linearly in a distal direction along the longitudinal axis 102 of the housing 3 together with the cover structure 210.

The engagement between the distally facing surface 32 of the engagement element 234 and the proximal surface 213 of the first cut-out 212 forces the lock member 230 to move linearly when the cover structure 210 moves to the retracted position shown in FIG. 13B.

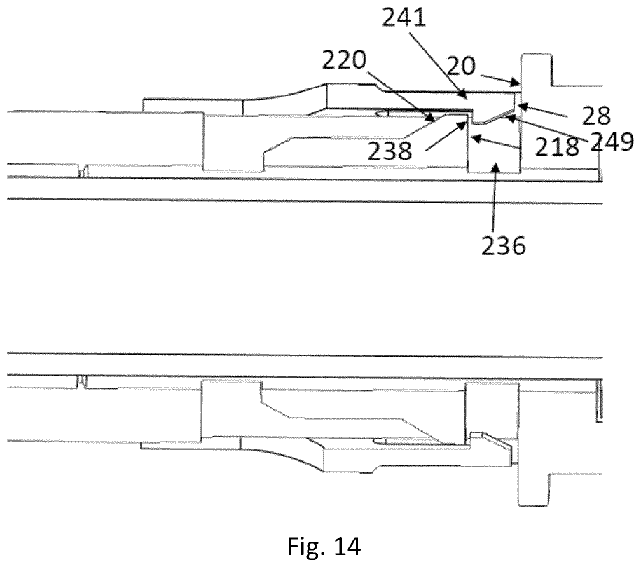

Once in the retracted position, the distal end 236 of the lock member 230 engages with a distal facing surface 218 of the carrier 216, better seen in FIG. 14 and FIG. 11. Turning specially to FIG. 14, and as discussed with reference to FIGS. 12A-B the distal end 236 of the lock member 230 comprises the proximally facing surface 238 that abuts against the distal facing surface 218 of the carrier 126. This engagement between the proximally facing surface 238 and the distal facing surface 218 locks the lock member 230 in place when the cover structure 210 subsequently moves in the proximal direction towards the extended position after the medicament delivery operation.

Further, the ramp surface 249 of the rib 241 is configured to slide on the inclined surface 220 of the carrier 216 when the lock member 230 moves in the distal direction when the cover structure moves towards the retracted position. This causes the rib 241 to flex radially away from the longitudinal axis to effectively be lifted in place in the engagement position shown in FIG. 12.

Turning to FIG. 13C where the cover structure 210 has moved to the extended position after a medicament delivery operation. This motion of the cover structure 210 causes a transition from the initial position to a locked position where the proximally facing surface 32 of the engagement element 232 abuts the distally facing surface 40 of the cover structure 210.

As the cover structure 210 moves from the retracted position, FIG. 13B, to the extended position in FIG. 13C, the inclined surface 240 of the engagement element 232 slides on a side wall 243 of the first cut-out opposite the proximal surface 213 of the first cut-out 212 so that the engagement element 232 flexes radially towards the longitudinal axis 102 to slide under the cover structure 210 more easily.

In the locked position shown in FIG. 13C the protrusion 234 is arranged in the second cut-out 214 comprising the distally facing 40 of the cover structure 210. The second cut-out 214 is distally arranged with respect to the first cut-out 212. In other words, the cover structure 210 moves linearly with respect to the lock member 230 when moving from the retracted position to the extended position.

As better seen in FIG. 14, in the locked position, a distal motion of the lock member 230 along the longitudinal axis 102 of the housing 3 is blocked by the stop surface 20 of the carrier 216 abutting the distally facing surface 28 of the lock member 230. The distal end 236 of the lock member 230 defines the distally facing surface 28 of the lock member that abuts against the stop surface 20 in the locked position.

Thus, the cover structure is prevented from again moving to the retracted position by the engagement with the engagement member 232 in the second cut-out 242 and by that the distally facing surface 28 of the lock member 230 that abuts against the stop surface 20 of the carrier for the needle 20.

A medicament delivery device (such as an autoinjector) may generally include various other components. For example, a sensor unit which may recognize injection events, such as the autoinjector inserted into an attachment portion of e.g., a pad, injection started, and injection ends, a memory unit which is configured to store the recorded data during the injection, a connectivity unit configured to transmit the stored data to a smart device or the network directly, a processing unit configured to control the entire system and processes the data before transmitting it, and/or user interface units that are configured to provide feedback to the patient, such as status LEDs, haptic, and/or audio feedback.

When the medicament delivery device is placed into the attachment portion, the sensors inside of the support pad are configured to recognize the event and give feedback to the patient via haptic/visual or audio elements.

When the injection finishes, the sensors are configured to recognize the event and give feedback to the patient again. Further, the collected data is stored in the memory unit and may be transmitted to the smart device/network via the connectivity unit after the injection event finishes.

The sensor can be one of or the combination of the following: a mechanical switch, a Hall-effect sensor, an accelerometer.

The mechanical switch, hall-effect sensor, or accelerometer can be used for detection of the insertion of the auto-injector into an injection port.

The accelerometer can be used for detecting injection events.

Possible wireless communication methods include Bluetooth and Cellular Networks.

Bluetooth connectivity requires a smart device to transmit the stored data to the network and it requires a pairing action between the support pad and the smart device before being able to use the supporting pad. But it's a cheaper alternative and it requires less space on PCB.

The cellular network does not require any pairing process, it can be used as a plug-n-play device, no prior setup is needed, but it's more expensive and it requires more space on PCB.

Depending on the requirements of the product any of those two technologies can be used.

Such processing units may comprise a logic circuit or control unit including a microprocessor, microcontroller, programmable digital signal processor or another programmable device. The processing circuitry may also, or instead, each include an application specific integrated circuit, a programmable gate array or programmable array logic, a programmable logic device, or a digital signal processor. Where the processing circuitry includes a programmable device such as the microprocessor, microcontroller or programmable digital signal processor mentioned above, the processor may further include computer executable code that controls operation of the programmable device.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A safety device for a medicament delivery device, the safety device comprising:
a housing extending in a longitudinal direction from a proximal end (3a) to a distal end relative a longitudinal axis;
a cover structure configured to surround a medicament delivery member at the proximal end of the housing, the cover structure is axially movable in relation to the housing along the longitudinal axis between a retracted position in which the medicament delivery member is exposed at the proximal end of the cover structure, and an extended position with the medicament delivery member covered by the cover structure;
a carrier immovably arranged within the housing configured to hold the medicament delivery member, the carrier comprises a stop surface facing the proximal end of the housing; and
a lock member arranged coaxially inside the cover structure within the housing, wherein the lock member comprises a distally facing surface, and an engagement element comprising a proximally facing surface, wherein a motion of the cover structure in a proximal direction towards the extended position after a medicament delivery operation causes a transition from an initial position where the proximally facing surface of the engagement element is spaced apart from a distally facing surface of the cover structure to a locked position where the proximally facing surface of the engagement element abuts the distally facing surface of the cover structure, wherein, in the locked position, a distal motion of the lock member along the longitudinal axis of the housing is blocked by the stop surface of the carrier abutting the distally facing surface of the lock member.

17. The safety device according to claim 16, wherein the lock member comprises a protrusion protruding outwards in a radial direction with respect to the longitudinal axis of the housing to engage with a guide of the cover structure.

18. The safety device according to claim 17, wherein the protrusion is configured to move into engagement with the guide when the cover structure moves to the retracted position.

19. The safety device according to claim 17, wherein the guide is helical, and wherein the protrusion is configured to move into engagement with the helical track of the guide.

20. The safety device according to claim 19, wherein the protrusion is initially in a recess or cut-out of the cover structure, wherein the protrusion moves from the recess or cut-out to the helical track when the cover structure initially moves in a distal direction from the extended position to the retracted position for a medicament delivery operation.

21. The safety device according to claim 17, wherein the protrusion comprises a proximally facing surface that is inclined with respect to the longitudinal axis of the housing.

22. The safety device according to claim 16, wherein engagement element is a stop rib that comprises the proximally facing surface and the distally facing surface of the lock member, wherein in the locked position, the stop rib is positioned between the stop surface of the carrier and the distally facing surface of the cover structure.

23. The safety device according to claim 18, wherein the guide is a groove or a ledge.

24. The safety device according to claim 16, wherein the engagement element comprises a protrusion, radially extending with respect to the longitudinal axis, wherein in the initial position the protrusion is arranged in a first recess or cut-out of the cover structure, and in the locked position the protrusion is arranged in a second recess or cut-out comprising the distally receiving surface of the cover structure, the second recess or cut-out being distally arranged with respect to the first recess or cut-out.

25. The safety device according to claim 24, wherein, in the initial position the protrusion of the engagement element is engaged in the first recess or cut-out of the cover structure so that when the cover structure initially moves towards the retracted position, the lock member moves linearly in a distal direction along the longitudinal axis of the housing together with the cover structure.

26. The safety device according to claim 25, wherein when the cover structure reaches the retracted position, a distal end of the lock member engages with a distal facing surface of the carrier.

27. The safety device according to claim 26, wherein the distal end of the lock member comprises a proximally facing surface that abuts against the distal facing surface of the carrier, when the cover structure moves in the proximal direction towards the extended position after the medicament delivery operation.

28. The safety device according to claim 26, wherein the distal end of the lock member defines the distally facing surface of the lock member that abuts against the stop surface in the locked position.

29. The safety device according to claim 25, wherein the protrusion of the engagement element comprises a distally facing surface that is inclined with respect to the longitudinal axis of the housing.

30. A medical delivery device comprising the safety device according to claim 16.
